(19) United States
(12) Patent Application Publication
(10) Pub. No.: US 2024/0272155 A1
(43) Pub. Date: Aug. 15, 2024

(54) MULTIPLY LABELLED PROTEIN FOR DETECTION ASSAYS

(71) Applicant: CAMBRIDGE MOLECULAR DIAGNOSTICS LTD, Cambridge (GB)

(72) Inventors: Gustavo Andrés Cerda Moya, Cambridge (GB); Benjamin Fraser Adam Shaw, Cambridge (GB)

(21) Appl. No.: 17/640,828

(22) PCT Filed: Sep. 7, 2020
(86) PCT No.: PCT/GB2020/052144

§ 371 Date: Mar. 6, 2022

(30) Foreign Application Priority Data
Sep. 6, 2019 (GB) 1912866.9

(51) Int. Cl. G01N 33/569; G01N 33/53; G01N 33/543

(52) U.S. Cl. CPC G01N 33/56911; G01N 33/5308; G01N 33/54346; G01N 33/54388; G01N 2469/10

(57) ABSTRACT

The present invention relates to methods, kits and devices for detecting a quantity of as biological target molecule. The invention is particularly relevant to techniques carried out on a flow based assay device. Each biological target molecule is labelled with a plurality of detectable nanoparticles, and may be detected on the device using an optical read-out.

### CROSS-REFERENCE TO RELATED APPLICATIONS

This application is a national phase application under 35 U.S.C. § 371 of International Application No. PCT/GB2020/052144, filed Sep. 7, 2020, which claims the benefit under 35 U.S.C. § 119(b) of GB Application No. 1912866.9, filed Sep. 6, 2019, each of which is herein incorporated by reference in its entirety.

### SEQUENCE LISTING

The Sequence Listing is hereby incorporated by reference in its entirety, including the file named 2726-4_ST25.txt, which is 11,890 bytes in size and was created on May 10, 2023, and submitted Jun. 20, 2023, which is herein incorporated by reference in its entirety.

### FIELD OF THE INVENTION

The present invention relates to methods, kits and devices for detecting a quantity of particular biomolecules, for example proteins or nucleic acids. The invention is particularly relevant to techniques carried out on a flow based assay device. The presence or absence of the biomolecules may be detected on the device using an optical read-out.

### BACKGROUND TO THE INVENTION

When detecting biomolecules in real-world situations, it is advantageous to accurately detect as few copies of the molecules as possible using simple instrumentation. Whilst nucleic acid samples can be amplified to increase the concentration of particular sequences, antibodies, proteins or other non-nucleic acid biomolecules can not be amplified, and hence the detection is limited by the number of such molecules appearing in the sample.

Nucleic acid amplification techniques usually require some form of hardware in order to ensure amplification occurs. Often the introduction of additional reagents is also required. Neither of these is desirable in situations where a fast and cheap read out on the presence or absence of a particular biomolecule in a sample is desired.

When performing genetic analysis, there is generally a need to amplify the number of copies in the sample, as the number present in the sample is generally too few to be detected. This can be done using, for example, thermocycling or isothermal amplification. PCR and Isothermal base amplification methods have been developed to allow the detection of low amounts of DNA or RNA. However this method can take about 90 min to generate results.

Thermocycling assays require hardware for heating and cooling as well as a means for detecting the presence of the amplified products.

Isothermal amplification techniques include SDA, LAMP, SMAP, HDA, EXPAR/NEAR, RPA, NASBA, ICAN, SMART. The reaction proceeds at a constant temperature using strand displacement reactions. Amplification can be completed in a single step, by incubating the mixture of samples, primers, DNA polymerase with strand displacement activity, and substrates at a constant temperature. Such methods typically amplify nucleic acid copies at least 10⁹ times in 15-60 minutes. However the requirement remains for a means for detecting the presence of the amplified material. Standard PCR based amplification can detect 1-10 molecules per reaction after 36 cycles (90-120 min). Isothermal amplification can achieve a similar performance to PCR based technologies.

Once the nucleic acid is amplified, a nucleic acid assay requires a secondary detection technology such as spectrophotometry or turbidity. However, such known techniques have drawbacks. Fluorescence detection requires labelling to allow fluorescence, making it expensive. The reagent SYBR green binds to DNA making it inherently carcinogenic; the Ames Test shows it to be both mutagenic and cytotoxic. Also SYBR green is not specific and attaches to any double stranded DNA thus increasing background signal. Turbidity measurements require expensive instrumentation to provide quantification.

In cases where non-nucleic acids are being detected, for example the presence of blood proteins, the sensitivity of the assay is dependent on the number of protein molecules in the sample. The number of molecules of the protein in the sample cannot be increased.

A common molecular detection technology is the lateral flow assay, where molecules are identified via antibody interactions on a support. Lateral flow assays are well known, and have been used for decades in a variety of assay platforms, for example home pregnancy tests. The basic flow assay has been used to develop a plethora of assays for clinical, veterinary, environmental, agricultural, bio-defensive and food-born pathogen screening applications. Strip assays are copiously adaptable and as such are commercially available for an extensive range of analytes including blood protein biomarkers, mycotoxins, viral and bacterial pathogens, as well as a whole range of nucleic acid detection products. However such assays are limited by the amounts of sample required as no amplification is carried out. Such assays have no amplification system, so the assays only work if sufficient amounts of the detected molecules are present in the test solution.

Since their introduction in the 1980s, lateral flow technologies have become important tools for point-of-care and home testing. They are commonly used to detect a broad array of targets such as HcG, infectious diseases and drugs of abuse and are also commonplace in veterinary testing, environmental testing and for monitoring analytes related to the human physiological condition. Initially tests provided a positive/negative result, but the development of reader technology and improvements in the materials and reagents has enabled a progression towards semi-quantitative and quantitative assays.

Lateral flow assays are essentially immunoassays which have been adapted to operate along a single axis to suit the format of a test strip. There are a number of variations of the technology that have been developed into commercial products, but they usually operate using the same basic concept. The technology is based on a series of capillary beds, such as pieces of porous paper or polymer. Each of these elements has the capacity to transport fluid, for example body fluids such as blood, saliva or urine or extracts thereof. A typical lateral flow assay test strip typically consists of the following components:

### 1. Sample Pad

An adsorbent pad onto which the test sample is applied. This acts as a sponge and holds an excess of sample fluid.

### 2. Conjugate or Reagent Pad

Once the sample pad is saturated the fluid migrates to a porous conjugate pad which contains antibodies specific to the target analyte conjugated to coloured particles (usually gold nanoparticles, or latex microspheres but in some instances fluorescent labels are used). When the sample fluid dissipates the matrix, it also dissolves the particles and in one combined, conveying action, the sample and conjugate mix flow through the porous structure. In this way, the analyte binds to the particles while migrating further along the test strip.

### 3. Reaction Membrane

This is typically a membrane onto which anti-target analyte antibodies are immobilised in a stripe that crosses the membrane to act as a capture zone or test line (a control zone will also be present, containing antibodies specific for the conjugate antibodies).

The reaction membrane has one or more areas (stripes) where a third molecule has been immobilized. By the time the sample-conjugate mix reaches these stripes, the analyte has been bound on the particle and the third ‘capture’ molecule in the stripe binds the complex. After a while, when more and more fluid has passed across the stripes, particles accumulate and the stripe-area changes colour. Typically there are at least two stripes: one (the control) that captures any particle and thereby shows that reaction conditions and technology worked fine, the second contains a specific capture molecule and only captures those particles onto which an analyte molecule has been immobilized.

### 4. Wick or Waste Reservoir

A further absorbent pad designed to draw the sample across the reaction membrane by capillary action. After passing the test stripes the fluid enters a final porous material, which simply acts as a waste container.

The components of the test strip are usually fixed to an inert backing material and may be presented in a simple dipstick format or within a plastic casing with a sample port and reaction window showing the capture and control zones.

The sensitivity of the test strip is limited by the amount of material in test solution. One molecule of the target analyte releases one antibody conjugate, which is attached to one detectable coloured particle. The sensitivity of the assay is therefore limited by the concentration of target in the original sample. Such assays are prone to false negative readings if the concentration of the biomolecule in the sample is too low.

### SUMMARY OF THE INVENTION

The present invention relates to methods and devices for improving the detection sensitivity of lateral flow assays. Prior art assays are limited in sensitivity as only one detectable moiety is linked to each sample molecule. The invention herein allows multiple detectable moieties to be captured per individual sample molecule, thereby increasing the level of directly detectable signal.

The concept of amplifying signal via binding of ligands that themselves have multiple binding sites to create branching structures has been used in various techniques. For example EP0354847 FIG. 1. drawing, where AFP is α-fetoprotein, B is biotin, SA is streptavidin and TG is thyroglobin:

The ultimate goal is to create as many sites for the gold conjugate to bind to that will be tethered in the detection zone.

Applicants have herein developed improved conjugates for use in assays, including lateral flow assays. By producing the structure in a different way to the art above, the applicants can create higher levels of signal amplification than would be possible with the prior art methods. In addition it can be used in more situations as it does not require the antibodies/ligands to undergo further chemical treatment.

The prior art methodology is dependent on use of biotinylated antibodies to amplify the signal. (As is the case in patents WO 00/25135 and EP0354847 A2 for example). Essentially antibodies/ligands must be treated with and coated in biotin. This process of NHS-Biotin linking has many downsides. It requires the proteins to be put through a process that can inhibit or reduce their binding function. Furthermore it relies on the availability of reactive groups on the protein surface that are able to react with the chemical linker (NHS-Biotin), which restricts the amount of groups that can be attached without affecting the function of the antibody.

Described herein is a method of creating fusion proteins with multiple subunits of small tags that will then act as docking sites for other specific tags/antibodies (examples include His (HHHHHH) (SEQ ID NO:1), FLAG (DYKDDDDK) (SEQ ID NO:2), E-tag (GAPVPYPDPLEPR) (SEQ ID NO:3), HA (YPYDVPDYA) (SEQ ID NO:4), Strept-tag (WSHPQFEK) (SEQ ID NO:5), Myc (EQKLISEEDL) (SEQ ID NO:6), S-tag (KETAAAKFERQHMDS) (SEQ ID NO:7), SH3 (STVPVAPPRRRRG) (SEQ ID NO:8), G4T (EELLSKNYHLENEVARLKK) (SEQ ID NO:9). Creating these fusion proteins rather than treating the proteins with factors such as biotin has many advantages.
1. It does not require the antibodies to go through further treatments that can inhibit their function
2. It allows control of the location of the tags to not inhibit the function of the antibodies.
3. More labels can be incorporated into each fusion protein without inhibiting function.
4. By introducing oligomerization peptides this allows each binding protein subunit to carry 4-7 times the amount of tags. (For example IgG antibody can carry approx. 2-10 biotin molecules. The fusion proteins start with 5-10 repeating subunits and then oligomerize into subunits that each will have 25-70 of the tags. Prior art branches will amplify signal by 2-10×. Each of the proteins described herein will amplify the signal 25-70×.

Described herein is a method to detect small numbers of target molecules in a short time using a polymeric molecular construct comprising a plurality of detectable nanoparticles, the polymeric molecular construct having multiple affinity binding sites wherein at least two of the affinity binding sites are each independently linked to a detectable nanoparticle. Each target molecule in the sample is thus labelled with multiple nanoparticles, thereby increasing the signal obtained from each molecule in the sample.

The multiple binding sites are within the protein strands rather than attached as appendages such as biotin. Thus the location and number of labels can be accurately designed and controlled.

Also described herein is a method to detect small numbers of target molecules in a short time using a labelled protein construct comprising a region that is specific for a target and a plurality of regions that are specific to detectable nanoparticles, the construct having at least two separate affinity binding sites independently linked to separate detectable nanoparticles.

The technology described herein relates to a polymeric molecular construct comprising a region that is specific for a target and a plurality of detectable nanoparticles, the polymeric molecule having multiple affinity binding sites wherein at least two of the affinity binding sites are each independently linked to a detectable nanoparticle.

The technology described herein relates to a labelled protein construct comprising a region that is specific for a target and a plurality of detectable nanoparticles, the construct having multiple affinity binding sites wherein at least two of the affinity binding sites are each independently linked to a detectable nanoparticle.

The technology described herein also relates to a lateral flow device for detecting the presence of a target, the device comprising:
(a) a sample loading area positioned at one end of the lateral flow device;
(b) an area comprising a polymeric molecular construct, wherein said polymeric molecular construct is bound to at least two detectable nanoparticles and has an affinity binding site specific to a target molecule in the sample, wherein the polymeric molecular construct is capable of wicking across at least a portion of the lateral flow device;
(c) an area comprising capture probes for the specific target and capture probes for the polymeric molecular construct, wherein said capture probes are immobilized on the lateral flow device; and
(d) absorbent material, wherein the absorbent material wicks an aqueous sample across the lateral flow device when the aqueous sample is added to the sample loading area.

The technology described herein also relates to a lateral flow device for detecting the presence of a target in a sample, comprising:
(a) a sample loading area positioned at one end of the lateral flow device;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.

The device can take the form of a test strip where the fluid flow occurs along a single axis. The device may also be referred to as a chip, where the strip is contained within a holder in order to aid handing of the strip. All of the chemicals and reagents required for detection of the target are immobilised onto a solid support surface which is then exposed to the fluid being tested for the target.

In an alternative method, the multiply labelled polymeric molecular construct or labelled protein construct can be mixed with the sample prior to application to the lateral flow device. In such devices where the sample and labelled polymer are pre-mixed, there is no need for the device to contain the area described in (b).

The flow assay can be a lateral flow assay, where fluid flows along a strip of porous material, or a vertical flow assay where the fluid passes through various sections under gravity or capillarity. The vertical and lateral flow can be combined.

The detection can be carried out without the need for any solution reagents as everything required can be immobilised on the surface of the device. Particular applications relate to the identification of biomolecules, for example proteins, antibodies or nucleic acid molecules. No further enzymes or molecules other than those mixed with the sample, or immobilised or adhered to the strip are required. For example the technology allows the detection of RNA without the need for reverse transcriptase, the detection of DNA without the need for polymerase based amplification and the detection of proteins without requiring enzymes or the substrates therefor. It therefore allows the detection of small amounts of molecules such as proteins, lipids, saccharides, metabolites, small molecules and chemicals.

The target can be any molecule for which the detection is desired. The target can be a protein. The target can be an antibody. The target can be a lipoprotein. The target can be a small molecule. The target can be a nucleic acid. The target can be DNA, RNA or modified forms thereof. The target may be derived directly from an organism, for example a virus, bacteria or other pathogen. The organism may be mammalian. Where the target is a nucleic acid, the method allows the specific detection of particular sequences, depending on the choice of target specific regions. The target nucleic acid strand may be single stranded or double stranded. In the labelled protein constructs described herein the plurality of regions may be selected from His, FLAG, E-tag, HA, Strept-tag, myc, S-tag, SH3, G4T.

### BRIEF DESCRIPTION OF THE DRAWINGS

FIG. 1 shows an exemplary polymeric conjugate or construct as described herein. A single target binding moiety (T) is labelled with multiple receptors (X′). The multiple receptors are available to bind to a moiety (X) each having further receptors (Y′). Receptors Y′ can bind to Y, each Y being attached to further multiple receptors Z′. Each Z′ can bind to a colloidal gold particle. Thus each target binding to a single (T) accumulates many colloidal gold particles. Whilst each colloidal gold particle may bind to many copies of Z′ per particle, each T will also bind to many colloidal particles. When molecule T, molecule X, molecule Y and molecule Z are constructed in sequential order, the signal is amplified when compared to a singly labelled T (e.g. up to hundreds of times in this case).

FIG. 2 shows a similar arrangement to FIG. 1. A single target binding moiety (T) is labelled with multiple receptors (shown as biotin for the sake of demonstration only). Each of the many receptors can be further labelled with molecules having many more receptors. The introduction of an anti-biotin or other receptor labelled colloidal gold particle produces a polymer with many gold particles per target binding moiety (T).

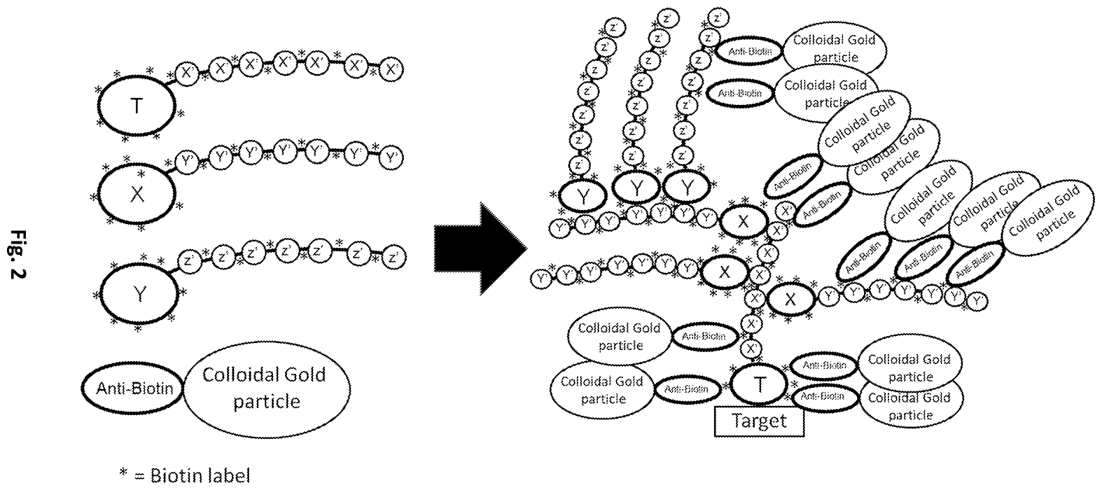

FIG. 3 shows an example of multi tagged molecules using homo oligomeric molecules: Molecule A is a homo heptamer (e.g. co-chaperonin GP31 from bacteriophage T4) covalently fused to molecule X, having a tail formed by Y′ tags positioned in tandem. As previous example in FIG. 1, oligomers increase the branching of molecules. In this case each homo heptamer molecule has 49 y′ tags, offering a 49 times amplification.

FIG. 4 shows an example of chemically tagged molecules using a branched molecule: Molecule X is covalently linked to a chemical polymer (e.g. poly-ethyleneimine, carboxymethylcellulose), this polymer can be further linked to other molecules (B), where B is a receptor tag.

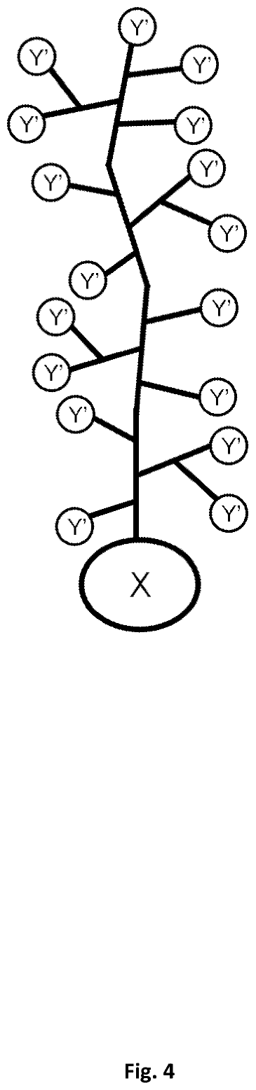

FIG. 5 shows an example of signal amplification using polymeric molecules: a modified oligonucleotide sequence is labelled with a peptide formed by x′ tags positioned in tandem using for example a covalent reaction. A further modified oligonucleotide is labelled with a peptide formed by y′ tags positioned in tandem using for example a covalent reaction. The complex formed by the hybridization of a target sequence in the presence of the two labelled oligonucleotides can be detected on a lateral flow device that contain Y molecules as test line. The interaction between the oligo complex can be detected using polymeric molecules containing binding moieties (X) and multiple nanoparticles to amplify the signal.

FIG. 6 shows an example of a strip assay or lateral flow device of the invention, where S represents a sample loading area, A¹, A² and A³ represent areas comprising a labelled protein construct, each B represents a buffer loading region and the test line comprises capture probes for a specific target.

### DETAILED DESCRIPTION OF THE INVENTION

The technology described herein relates to a polymeric molecular construct comprising a plurality of detectable nanoparticles. The technology described herein also relates to a labelled protein construct comprising a region that is specific for a target and a plurality of regions that are specific to detectable nanoparticles. Each construct has a region that is specific for a target. Each construct has a plurality of detectable nanoparticles. The plurality of nanoparticles can be attached via ligand binding, where suitable ligands act as affinity binding sites. Thus each polymeric molecular construct or labelled protein construct has multiple affinity binding sites within the protein sequence. Each of the multiple affinity binding sites can act independently. Therefore multiple affinity binding sites will act to bind to multiple separate detectable nanoparticles. At least two of the affinity binding sites will be independently linked to a separate detectable nanoparticle, thereby giving a polymeric molecule having at least two detectable nanoparticles on the same polymeric molecule. The at least two affinity binding sites may be independently linked to separate detectable nanoparticles.

The use of such constructs means that each individual target molecule will be labelled with multiple nanoparticles, thereby increasing the sensitivity of the detection.

The term comprising is used to indicate that the polymeric molecular construct or labelled protein construct is an assembly of multiple molecular species, and is not necessarily a single covalently linked molecule. Depending on the level of signal amplification, the construct can optionally be a single chain linked to multiple particles, or can be a single chain linked to further chains, where each further chain carries the particles (as shown in the figures), thereby obtaining a level of branching which can optionally be repeated using suitable affinity binding regions. Thus the labelled nanoparticles may be bound directly to the peptides, or may be attached via other branched linking groups, for example other peptides.

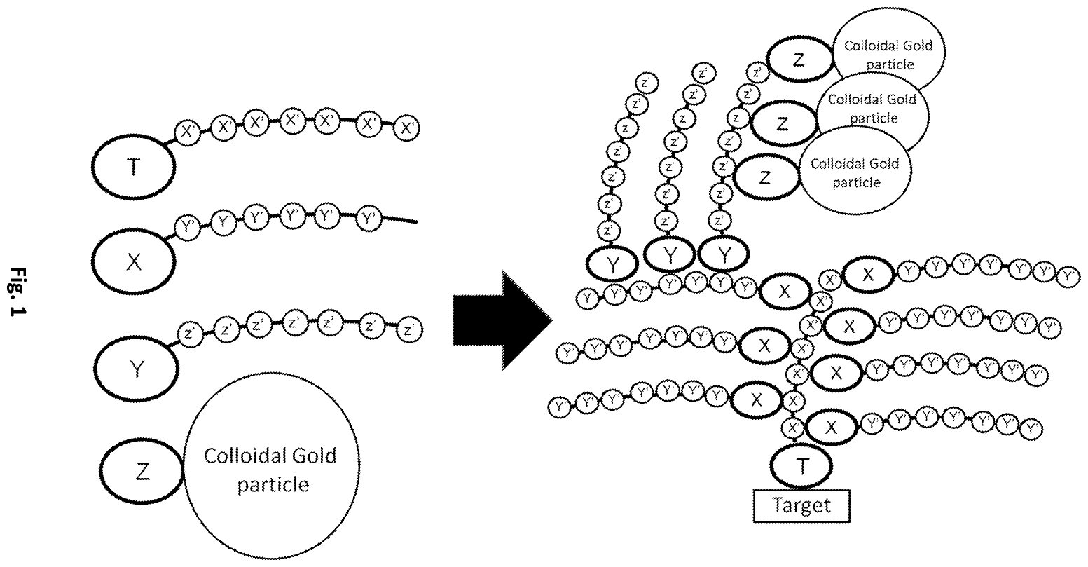

For example, FIG. 1 shows an exemplary polymeric conjugate or construct as described herein. A single target binding moiety (T) is labelled with multiple receptors (X′) (the affinity binding sites). The multiple affinity binding sites are available to bind to a moiety (X) each having further receptors (Y′). Receptors Y′ can bind to Y, each Y being attached to further multiple receptors Z′. Each Z′ can bind to a colloidal gold particle. Thus each target binding to a single (T) accumulates many colloidal gold particles via a plurality of binding events. Whilst each colloidal gold particle may bind to many copies of Z′ per particle, each T will also bind to many colloidal particles. When molecule T, molecule X, molecule Y and molecule Z are constructed in sequential order, the signal is amplified when compared to a singly labelled T (e.g. up to hundreds or thousands of times).

The detectable nanoparticles may be for example antibody coated. The polymeric molecule or labelled protein may contain a plurality of antigens such that each polymeric molecule or labelled protein can bind to more than one nanoparticles (i.e. the affinity sites can be antigens specific to particularly coated nanoparticles). The antigens can be for example peptide regions which can appear repeatedly in particular polymers or proteins. Thus for example the polymers or proteins can be repeating strings of particular amino acids where the amino acids act as antigens (receptors) to antibodies coated on the nanoparticles.

In one embodiment, the polymeric molecular construct comprises:
(a) a region specific for a target from a sample;
(b) multiple affinity binding sites; and
(c) a plurality of detectable nanoparticles, wherein said detectable nanoparticles are antibody coated and said antibodies are linked to the affinity binding sites of the polymeric molecule such that each polymeric molecule is linked to a plurality of detectable nanoparticles in a linear configuration.

In one embodiment, the labelled protein construct comprises:
(a) a region specific for a target from a sample;
(b) multiple affinity binding sites; and
(c) a plurality of detectable nanoparticles, wherein said detectable nanoparticles are antibody coated and said antibodies are linked to the affinity binding sites such that each protein molecule is linked to a plurality of detectable nanoparticles.

In one embodiment, the polymeric molecular construct comprises:
(a) a region specific for a target from a sample;
(b) multiple first affinity binding sites for a binding species having further affinity binding sites which may be the same or different;
(c) more than one binding species attached to the first affinity sites; and
(d) a plurality of detectable nanoparticles, wherein said detectable nanoparticles are antibody coated and said antibodies are linked to the further affinity binding sites of the binding species such that each of the multiple binding species is linked to a plurality of detectable nanoparticles, and each construct is in a branched configuration.

In one embodiment, the labelled protein construct comprises:
(a) a region specific for a target from a sample;
(b) multiple first affinity binding sites for a binding species having further affinity binding sites which may be the same or different;
(c) more than one binding species attached to the first affinity sites; and
(d) a plurality of detectable nanoparticles, wherein said detectable nanoparticles are antibody coated and said antibodies are linked to the further affinity binding sites of the binding species such that each of the multiple binding species is linked to a plurality of detectable nanoparticles, and each construct is in a branched configuration.

In one embodiment, the polymeric molecular construct or labelled protein construct has between 100 and 10,000 detectable nanoparticles.

In one embodiment, the polymeric molecular construct or labelled protein construct has between 500 and 5000 detectable nanoparticles.

In one embodiment, the polymeric molecular construct or labelled protein construct has greater than 20 detectable nanoparticles.

In one embodiment, the polymeric molecular construct or labelled protein construct has greater than 50 detectable nanoparticles.

In one embodiment, the polymeric molecular construct or labelled protein construct comprises a plurality of detectable nanoparticles selected from: gold, iron, copper, silver, silver nucleated gold, platinum, carbon, cellulose beads.

In one embodiment, the polymeric molecular construct or labelled protein construct comprises a plurality of detectable gold nanoparticles.

In one embodiment, the polymeric molecular construct or labelled protein construct comprises a plurality of detectable carbon nanoparticles.

In one embodiment, the polymeric molecular construct or labelled protein construct comprises a plurality of detectable silver nucleated gold nanoparticles.

In one embodiment, the polymeric molecular construct or labelled protein construct comprises a plurality of detectable platinum nanoparticles.

In one embodiment, the polymeric molecular construct or labelled protein construct comprises a plurality of detectable cellulose nanobeads.

The nanoparticles are typically sub-micrometer in size. Typical colloidal nanoparticles can be 5-100 nanometers in size. The size and shape of the particles is unimportant, but the particles are often spherical, but can be cylindrical or any other shape. A larger increase in signal can be obtained from smaller nanoparticles. The size of the particles may be between 5 and 20 nm. The size of the particles may be 5, 10, 15 or 20 nm.

The term affinity binding site refers to a region of amino acid/peptide sequence. The affinity binding site can be a region of amino acid/peptide sequences specific to a particular antibody. The affinity binding site can be one or more of Glu-Glu-tag, HA-tag, Myc-tag, GCN4-tag, Anti-SH3 protein, ubiquitin. In the polymeric molecular constructs or labelled protein constructs described herein the plurality of regions can be selected from His, FLAG, E-tag HA, Strept-tag, myc, S-tag, SH3, G4T.

A list of examplary peptide affinity binding sites is below:

Alfa-tag
(SEQ ID NO: 10)
(SRLEEELRRRLTE)
Avi-tag
(SEQ ID NO: 11)
(GLNDIFEAQKIEWHE)
C-tag
(SEQ ID NO: 12)
(EPEA)
Calmodulin-tag
(SEQ ID NO: 13)
(KRRWKKNFIAVSAANRFKKISSSGAL)
Dogtag
(SEQ ID NO: 14)
(DIPATYEFTDGKHYITNEPIPPK)
E-tag
(SEQ ID NO: 3)
(GAPVPYPDPLEPR)
FLAG
(SEQ ID NO: 2)
(DYKDDDDK)
G4T
(SEQ ID NO: 9)
(EELLSKNYHLENEVARLKK)
HA
(SEQ ID NO: 4)
(YPYDVPDYA)
His
(SEQ ID NO: 1)
(HHHHHH)
Isopeptag
(SEQ ID NO: 15)
(TDKDMTITFTNKKDAE)
Myc
(SEQ ID NO: 6)
(EQKLISEEDL)
NE-Tag
(SEQ ID NO: 16)
(TKENPRSNQEESYDDNES)
Poly Glutamate-tag
(SEQ ID NO: 17)
(EEEEEEE)
Poly Arginine-tag
(SEQ ID NO: 18)
(RRRRRRR)
Rho1D4-tag
(SEQ ID NO: 19)
(TETSQVAPA)
SBP-tag
(SEQ ID NO: 20)
(MDEKTTGWRGGHVVEGLAGELEQLRARLEHHPQGQREP)
Sdytag
(SEQ ID NO: 21)
(DPIVMIDNDKPIT)
SH3
(SEQ ID NO: 8)
(STVPVAPPRRRRG)
Snooptag
(SEQ ID NO: 22)
(KLGDIEFIKVNK)
Softag 1
(SEQ ID NO: 23)
(SLAELLNAGLGGS)
Softag 3
(SEQ ID NO: 24)
(TQDPSRVG)
Spot-tag
(SEQ ID NO: 25)
(PDRVRAVSHWSS)
Spytag
(SEQ ID NO: 26)
(AHIVMVDAYKPTK)
S-tag
(SEQ ID NO: 7)
(KETAAAKFERQHMDS)
Strep-tag
(SEQ ID NO: 5)
(WSHPQFEK)
T7tag
(SEQ ID NO: 27)
(MASMTGGQQMG)
TC-tag
(SEQ ID NO: 28)
(EVHTNQDPLD)
Ty-tag
(SEQ ID NO: 29)
(CCPGCC)
VSV-tag
(SEQ ID NO: 30)
(YTDIEMNRLGK)
Xpress-tag
(SEQ ID NO: 31)
(DLYDDDDK)

The binding species can be an antibody. The binding species can be any species corresponding to the above-mentioned binding sites. The binding species can be Anti-Glu-Glu-tag, Anti-HA antibody, Anti-Flag antibody, Anti-Myc antibody, anti-GCN4 antibody, Anti-SH3 protein, ubiquitin binding proteins.

The target can be any molecule for which the detection is desired. The target can be a nucleic acid, for example DNA, RNA or modified forms thereof. The target can be a particular protein. The target may be a drug or a drug metabolite.

The target may be derived directly from an organism, for example a virus, bacteria or other pathogen. The target may be from a eukaryotic source, a microorganism, a virus, or a microbiome. The source may be mammalian. The target may be a particular sequence of nucleic acid, DNA, RNA or protein. The target nucleic acid may be single stranded or double stranded.

In one embodiment, the eukaryotic source is selected from algae, protozoa, fungi, slime molds and/or mammalian cells. In one embodiment, the microorganism or virus is selected from Escherichia, Campylobacter, Clostridium difficile, Enterotoxigenic E. coli (ETEC), Enteroaggregative Escherichia coli (EAggEC), Shiga-like Toxin producing E. coli, Salmonella, Shigella, Vibrio cholera, Yersinia enterocolitica, Adenovirus, Norovirus, Rotavirus A, Cryptosporidium parvum, Entamoeba histolytica, Giardia lamblia, Clostridia, Methicillin-resistant Staphylococcus aureus MRSA, Klebsiella pneumonia, flu, Zika, dengue, chikungunya, West Nile virus, Japanese encephalitis, malaria, HIV, H1N1, and Clostridium difficile resistant organisms.

In one embodiment, the sample is a biological sample from a subject. The biological sample from the subject can be selected from stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool water, pancreatic juice, lavage fluids from sinus cavities, bronchopulmonary aspirates, blastocyl cavity fluid, and umbilical cord blood.

The technology described herein also relates to a lateral flow device for detecting the presence of a target, the device comprising:
(a) a sample loading area;
(b) an area comprising a polymeric molecular construct, wherein said polymeric molecular construct is bound to at least two detectable nanoparticles and has an affinity binding site specific to a target molecule in the sample, wherein the polymeric molecular construct is capable of wicking across at least a portion of the lateral flow device;
(c) an area comprising capture probes for the specific target and capture probes for the polymeric molecular construct, wherein said capture probes are immobilized on the lateral flow device; and
(d) absorbent material, wherein the absorbent material wicks an aqueous sample across the lateral flow device when the aqueous sample is added to the sample loading area.

The technology described herein also relates to a lateral flow device for detecting the presence of a target in a sample, comprising:
(a) a sample loading area;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.

In one embodiment, the lateral flow device comprises a solid support. The solid support can be selected from glass, paper, nitrocellulose and thread. The lateral flow assay test strip may consist of the following components:

An adsorbent pad onto which the test sample is applied. This acts as a sponge and holds an excess of sample fluid.

A conjugate or reagent pad. Once the sample pad is saturated the fluid migrates to a porous conjugate pad which contains regions specific to the target analyte conjugated to the multiply particle labelled polymeric molecular constructs. When the sample fluid dissipates the matrix, it also dissolves the polymeric molecular constructs and in one combined, conveying action, the sample and conjugate mix flow through the porous structure. In this way, the analyte binds to the particles while migrating further along the test strip.

A reaction membrane. This is typically a membrane onto which anti-target analytes are immobilised in a stripe that crosses the membrane to act as a capture zone or test line (a control zone can also be present, containing analytes specific for the multiply labelled polymer).

The reaction membrane has one or more areas (stripes) where a third molecule has been immobilized. By the time the sample-conjugate mix reaches these stripes, the analyte has been bound on the particles and the third ‘capture’ molecule in the stripe binds the complex. After a while, when more and more fluid has passed across the stripes, particles accumulate and the stripe-area changes colour. Typically there are at least two stripes: one (the control) that captures any particle and thereby shows that reaction conditions and technology worked fine, the second contains a specific capture molecule and only captures those particles onto which an analyte molecule has been immobilized.

A wick or waste reservoir. A further absorbent pad designed to draw the sample across the reaction membrane by capillary action. After passing the test stripes the fluid enters a final porous material, which simply acts as a waste container.

The components of the test strip are usually fixed to an inert backing material and may be presented in a simple dipstick format or within a plastic casing with a sample port and reaction window showing the capture and control zones.

In one embodiment, the device can take the form of a test strip where the fluid flow occurs along a single axis. The device may also be referred to as a chip, where the strip is contained within a holder in order to aid handing of the strip. All of the chemicals and reagents required for detection of the target are immobilised onto a solid support surface which is then exposed to the fluid being tested for the target.

In one embodiment, flow assay can be a lateral flow assay, where fluid flows along a strip of porous material, or a vertical flow assay where the fluid passes through various sections under gravity or capillarity. The vertical and lateral flow can be combined.

In one embodiment, detection can be carried out without the need for any solution reagents as everything required can be immobilised on the surface of the device. Particular applications relate to the identification of protein or nucleic acid molecules. No further enzymes or molecules other than those immobilised are required. For example the technology allows the detection of RNA without the need for reverse transcriptase or the detection of DNA without the need for polymerase based amplification. It also allows the detection of small amounts of other molecules such as lipoproteins, lipids, saccharides, metabolites, small molecules and chemicals.

In one embodiment, the measurement may be a simple end point detection (is the target present; yes or no), or may involve an element of quantitative analysis. For quantitative analysis, the device can be coupled to a suitable reader allowing a direct measurement of the signal intensity in the detection zone. This can be correlated to the number of molecules present in the target sample. For semi-quantitative analysis, the detection zone can be calibrated to bind different amounts of colour particles (for example gold colloidal stained protein, bound to a printed antibody).

For end point or semi-quantitative detection, the detection can be carried out using the human eye, rather than requiring any further hardware to read the result.

In one embodiment, detection can be carried out in multiple zones or lines. For example, for semi quantitative detection different lines with different amount of trapping molecules can be printed (e.g. a first line containing 25 ng/cm, a second line containing 250 ng/cm, a third line containing 2.5 μg/cm and so on). Therefore, considering the molecular weight of the trapping molecule, the accumulation of colour on the stripes will be a reflection of the amount of target on the sample (i.e. if sample contains 1-10 target molecules, only first line will accumulate the colour. If sample contains 10-100 target molecules, first and second lines will accumulate the colour. If sample contains 100-1,000 target molecules, first, second and third lines will accumulate the colour etc. Thus the quantification can be carried out using the different bands where the different bands have different responses depending on the amount of detectable marker in the fluid.

The technology described herein also relates to a method for detecting the presence of a target in a biological sample from a subject, comprising:
i) adding the sample to a sample loading area of a lateral flow device, wherein said device comprises:
   (a) a sample loading area;
   (b) an area comprising a polymeric molecular construct, wherein said polymeric molecular construct is bound to at least two detectable nanoparticles and has an affinity binding site specific to a target molecule in the sample, wherein the polymeric molecular construct is capable of wicking across at least a portion of the lateral flow device;
   (c) an area comprising capture probes for the specific target and capture probes for the polymeric molecular construct, wherein said capture probes are immobilized on the lateral flow device; and
   (d) absorbent material, wherein the absorbent material wicks an aqueous sample across the lateral flow device when the aqueous sample is added to the sample loading area.
ii) determining from the presence of detectably labelled probe in (c) the presence of the target in the sample.

The technology described herein also relates to a method for detecting the presence of a target in a biological sample from a subject, comprising:
i) adding the sample to a sample loading area of a lateral flow device, wherein said device comprises:
(a) a sample loading area;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.
ii) determining from the presence of detectably labelled probe in (c) the presence of the target in the sample.

The technology described herein also relates to a method for detecting the presence of a target in a biological sample from a subject, comprising:
i) mixing a sample with a polymeric molecular construct, wherein said polymeric molecular construct is bound to at least two detectable nanoparticles and has an affinity binding site specific to a target molecule;
ii) adding the sample to a sample loading area of a lateral flow device, wherein said device comprises:
(a) a sample loading area;
(b) an area comprising capture probes for the specific target and capture probes for the polymeric molecular construct, wherein said capture probes are immobilized on the lateral flow device; and
(c) absorbent material, wherein the absorbent material wicks an aqueous sample across the lateral flow device when the aqueous sample is added to the sample loading area.
ii) determining from the presence of detectably labelled probe in (b) the presence of the target in the sample.

The technology described herein also relates to a method for detecting the presence of a target in a biological sample from a subject, comprising:
i) mixing a sample with a labelled protein construct, wherein said construct is bound to at least two detectable nanoparticles and has an affinity binding site specific to a target molecule;
ii) adding the sample to a sample loading area of a lateral flow device, wherein said device comprises:
(a) a sample loading area;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.
iii) determining from the presence of detectably labelled probe in (c) the presence of the target in the sample.

In one embodiment, the present technology can be used for fast detection of the presence of many targets, for example interleukins, hormones, oncogenes (as protein or nucleic acid), pathogens (as protein or nucleic acid), virus (as protein or nucleic acid), drugs, toxins, metabolites. The target can be mycoplasma, for example for identification of cell line contamination. The target can be a coronavirus. Assays and conjugates as described herein can be used for detection of virus infection in mammalian samples.

Fields in which the technology may be used include pathogen identification and contaminant tracing; forensic analyses; food industry; soil analyses; agriculture; aquaculture etc.

Also disclosed is a kit of reagents for detecting a target, the kit containing a device as described above and a buffer solution into which a biological sample can be added. The kit may further include instructions for use of the kit.

WO2016/203272 describes a flow assay where molecules are amplified during the assay. The use of the labelled constructs described herein can also be used within such an amplification assay.

The technology described therein releases a small number of molecules, which triggers an amplification of the signal inducing a cascade or “domino effect” that amplifies the signal in a near-exponential manner. No reagents are required from solution, the amplification cascade is initiated simply by applying a test sample to the device. In the following description, the term displaced refers to the process of causing release from a surface without breaking chemical bonds. Displacement is a spontaneous process caused by the presence of the target analyte, rather than a chemical reaction that requires another reagent to enable the release.

Described herein is a device for detecting the presence of a target analyte in a fluid, the device comprising:
i) a displacement area having a first immobilised marker, for example having a protease or other enzyme, which can be displaced by the presence of the target analyte to produce a first released marker, wherein the displacement occurs without breaking covalent bonds, for example by denaturing a nucleic acid duplex;
ii) one or more signal amplification areas having further immobilised markers which can be released by the presence of the first released (enzymatic) marker to produce a detectable marker, wherein the detectable marker is a polymeric molecular construct comprising a region that is specific for a target and a plurality of detectable nanoparticles, the polymeric molecular construct having multiple affinity binding sites wherein at least two of the affinity binding sites are each independently linked to a detectable nanoparticle; and
iii) one or more detection areas which can identify the presence of the detectable marker;
wherein the displacement area, signal amplification area(s) and detection area(s) are connected such that fluid can flow from the displacement area through the signal amplification area(s) and into the detection area(s).

Described herein is a device for detecting the presence of a target analyte in a fluid, the device comprising:
i) a displacement area having a first immobilised marker, for example having a protease or other enzyme, which can be displaced by the presence of the target analyte to produce a first released marker, wherein the displacement occurs without breaking covalent bonds, for example by denaturing a nucleic acid duplex;
ii) one or more signal amplification areas having further immobilised markers which can be released by the presence of the first released (enzymatic) marker to produce a detectable marker, wherein the detectable marker is a labelled protein construct comprising a region that is specific for a target in the sample and a plurality of regions that are specific to detectable nanoparticles, the construct having at least two separate affinity binding sites independently linked to separate detectable nanoparticles; and
iii) one or more detection areas which can identify the presence of the detectable marker;
wherein the displacement area, signal amplification area(s) and detection area(s) are connected such that fluid can flow from the displacement area through the signal amplification area(s) and into the detection area(s).

The presence of one target molecule in the displacement area produces the release of one molecule that will travel with the flow of the sample. When the released molecule (or first released marker) gets into contact with the next section, the signal amplification area, it will trigger the release of more molecules, for example 80-200 molecules each having detectable markers, that will also travel with the fluid flow and will allow the release of more molecules, for example 80-200 molecules per each molecule released on the previous step. This will produce the same reaction in the following step and so on, giving a near-logarithmic amplification in the amount of detectable marker molecules. The amplification modules can be used multiple times in series allowing high sensitivity. The cascade can be achieved by chemical cleavage of molecules, by the use of proteases or enzymatically active molecules.

Once the sample has reached the detection area, there will be enough released molecules that an enzymatic reaction or accumulation of colour reaction product can be used to detect the signal, even if the amount of target analyte applied in the fluid sample was very low.

In order to detect a single applied molecule, amplification to the marker molecules can allow for, for example, at least 106 or more copies of the detectable marker. Such an amount is readily detectable. The number of detectable markers may be for example 10⁸ or higher. The number of detectable markers may be for example 10⁹ or higher. The number of detectable marker can be adjusted by the size and number of the amplification zones. Larger amplification zones mean that the sample takes longer to flow through the zone, thereby giving more time to catalyse release of the detectable markers.

In contrast to the displacement, where no covalent bonds are broken, the amplification cascade can be carried out by cleaving material from the surface via chemical bond cleavage. The chemical covalent bond cleavage can be carried out using enzymes or other catalysts, the presence of which is only occasioned should they be displaced from the displacement area. Thus the amplification cascade can be a catalysed reaction, whereas the displacement using the target analyte is a non catalysed reaction.

The present description of this technology uses as an exemplar specific peptidases on the cascade to amplify the signal. However to achieve the same result, other enzymatically active molecules can be used such as nucleases, lipases, disaccharidases, polysaccharides, kinases, phosphorylases, methylases, sumorilases, ubiquitin deacetylases.

### EXAMPLES

Examples of compounds having many labels are shown below:

### Example 1

### Expression of Recombinant Molecules Fused to Tags Positioned in Tandem.

Fab anti-GCN4 antibody sequence was cloned in frame with 7xMyc tag (7xGSKSGEQKLISEEDL) (SEQ ID NO:32) and a 6xHis tag for purification (FIG. 1). Fab anti-Myc antibody sequence was cloned in frame with 10xSH3 tag (10xSTVPVAPPRRRRG) (SEQ ID NO:8) and a 6xHis tag for purification (FIG. 1). Anti-SH3 protein sequence was cloned in frame with 6xHis tag for purification (FIG. 1). Recombinant proteins were expressed using New England Biolab Shuffle cells and purified using NTA-agarose and further purified using AKTA gel filtration P-200.

Poly tag proteins (e.g. KK-6xHis-7xFlag, KK-6xhis-7xG4tag and KK-6xHis7xHA) were cloned in to T7 expression vector. Recombinant proteins were expressed using Thermo Scientific BL21(DE3) cells and purified using NTA-agarose and further purified using AKTA gel filtration P-200.

### Expression of Recombinant Self Homo Oligomeric Molecules.

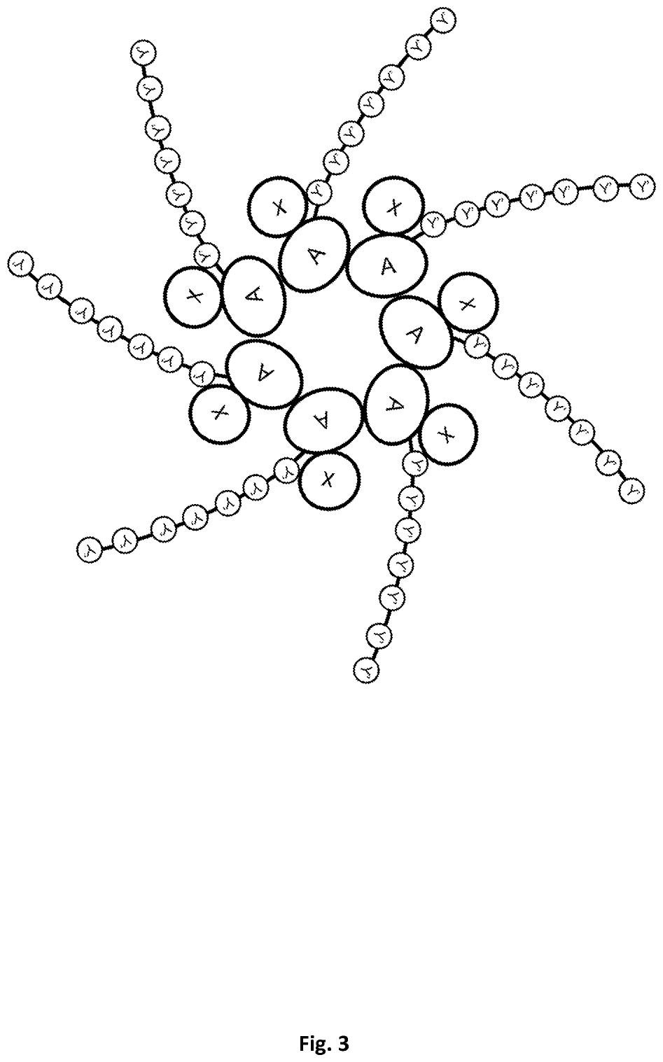

Anti-GFP nanobody sequence was cloned in frame homo heptamer co-chaperonin GP31 protein from bacteriophage T4 (FIG. 3) and 7xGCN4tag (7xGSKSGEELLSKNYHLENEVARLKK) (SEQ ID NO:34), including a 6xHis tag for purification. Homo oligomer molecules have also been made fusing tetramer molecules such as avidin and streptavidin.

### Covalent Binding of Molecules to Branched Molecule.

Single amino group of branched molecule PEI (Poly-ethyleneimine, Sigma 408700) were transformed into maleimide groups by reacting with crosslinker SMCC (Succinimidyl trans-4-(N-maleimidylmethyl) cyclohexane-1-carboxylate) using 1:1.5 molar ratio. Thiolation of Anti-MYC antibody was performed using SPDP (Succinimidyl 3-(2-pyridyldithio) propionate). Thiol modified Anti-MYC antibody was reduced using TCEP before were added drop by drop to single-maleimide derived PEI. The result of this reaction was a fusion between Anti-Myc antibody to PEI, this was subsequently purified using AKTA gel filtration P-200. The remaining amino groups of Anti-Myc-PEI were subsequently transformed into maleimide groups by reacting with crosslinker SMCC using 1:200 molar ratio. Thiolation of SH3 peptide (MGSGSTVPVAPPRRRRG) (SEQ ID NO:33) was performed using SPDP, alternatively Cysteine SH3 peptides (MCGSGSTVPVAPPRRRRG) (SEQ ID NO:35) was used with less efficiency. Thiol modified SH3 peptide or Cysteine SH3 peptides were reduced using TCEP before they were added drop by drop to maleimide derived Anti-Myc-PEI. The result of this reaction is Anti-Myc that carries 15-25 SH3 tags.

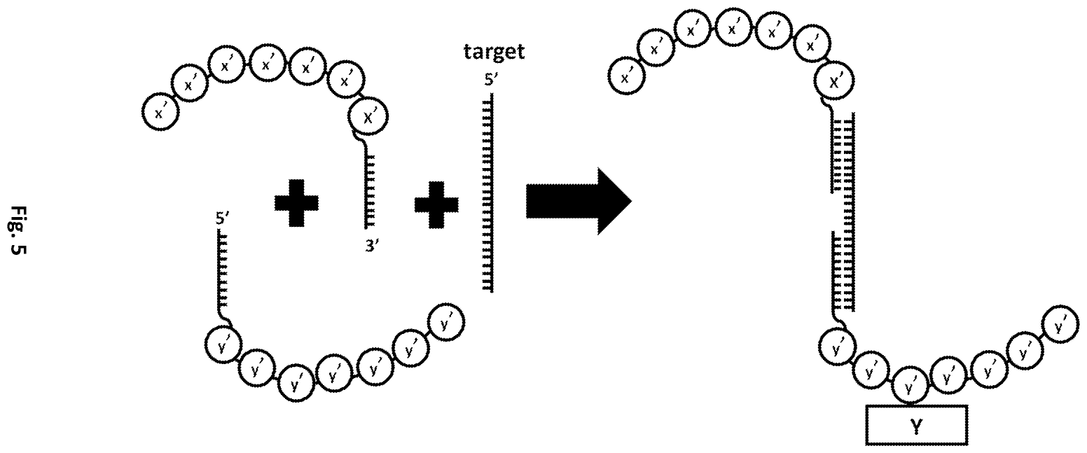

### Increasing Sensitivity of Oligo Chromatography FIG. 5:

Poly tag proteins (e.g. KK-6Xhis-7xFLAG, KK-6xhis-7xG4tag and KK-6xHis7xHA) were transformed into maleimide groups by reacting with crosslinker SMCC (Succinimidyl trans-4-(N-maleimidylmethyl) cyclohexane-1-carboxylate). Thiol modified oligos (ATCTGTCTATTTCGTTCATC-3′Thiol (SEQ ID NO:36), 5′Thiol-CCAATGCTTAATCAGTGAG (SEQ ID NO:37), CAGTTCTTCACCT TTGCCAAC-3′Thiol (SEQ ID NO:38 and 5′Thiol-CACCAGAATCAGTGCACAAC (SEQ ID NO:39)) were reduced using DTT before were added drop by drop to maleimide derived tag protein. These reactions produce ATCTGTCTATTTCGTTCATC-7xG4Tag (SEQ ID NO:36), 7xHA-CCAATGCTTAATCAGTGAG (SEQ ID NO:37), CAGTTCTTCACCTTTGCCAAC-7xG4Tag (SEQ ID NO:38) and 7xFLAG-CACCAGAATCAGTGCACAAC (SEQ ID NO:39) linked oligos (as described on FIG. 5). Similar results can be achieved by the thiolation of amino modified oligos using SPDP (Succinimidyl 3-(2-pyridyldithio) propionate).

Labelled oligos were hybridized to target sequence 5′ TGGATGAACGAAATAGACA GATCGCTGAGATAGGTGCCTCACTGATTAAGCATTGGTAAGGT (SEQ ID NO:40) and 5′ GTTGGCAAAGGTGA AGAACTGTTTACCGGCGTTGTGCACTGATTCTGGTG (SEQ ID NO:41) before loading the mix on to a lateral flow device that has been previously printed with 12CA5 anti-HA antibody and Anti-FlagM2 antibody. Hybridized oligos were detected using a mix of anti-GCN4-7xMyc, Anti-Myc-10xSH3 and Anti-SH3 Colloidal Gold. Alternatively Anti-Myc-PEI-20xSH3 was used replacing Anti-Myc-10xSH3.

### Preparation of Colloidal Gold Protein Conjugates.

Colloidal gold solution was prepared following method from Turkevich et al (Discuss. Faraday Soc., 1951, 11, 55-75) and G. Frens (Phys. Sci., 1973, 241, 20-22). 200 ml of 0.01% HAuCl4 in Milli-Q water was stirred vigorously and heated till boiling under reflux conditions. Depending on designated particle size sodium citrate (1% aqueous solution) was added very quickly while the solution was stirred and boiled vigorously. After about 1 minute the light yellow solution loses colour completely before changing colour to deep blue and finally dark red. To make sure the reaction had finished completely, the solution was allowed to boil for another 10 min. After cooling to room temperature pH was adjusted with 1 ml of 0.2 M K₂CO₃ solution. The desired conjugate protein, such as Anti-SH3 protein, was diluted in 10 ml of PBS, and added in small volumes to 200 ml colloidal gold with continuous stirring. After mixing the conjugation reaction was left for 30 minutes at room temperature. The appropriate amount of conjugate protein, such as Anti-SH3 protein, was empirically calculated by performing a series dilution and measuring the protection of colloidal gold to 10% Sodium Chloride. After conjugation Anti-SH3 Colloidal gold solution was precipitated at 8000 g for 30 min and resuspended in 20 ml of PBS 0.002% Tween-20.

### Lateral Flow Test for Detection of Ampicillin Resistance.

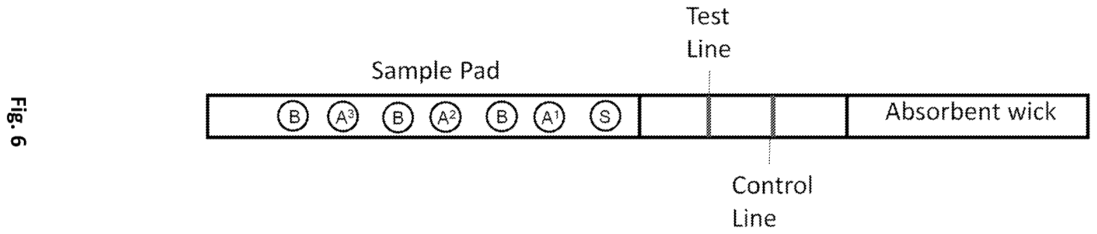

Lateral flow device was printed with two lines on the nitrocellulose membrane (Test Line using 1 μg/cm of 12CA5 anti-HA antibody and Positive control using 0.5 μg/cm of Anti-Flag M2 antibody) (FIG. 6). Three solutions containing 2% sucrose, 1%BSA, 0.5% and either Anti-GCN4-7X Myc, Anti-Myc-10XSH3 or Anti-SH3 Colloidal Gold were prepared, the solutions were printed on the cellulose sample pad on position A¹, A² and A³ respectively (FIG. 6), lateral flow strips where dried for 2 hours at 37° C.

20 μL of bacteria was mixed with 20 μL lysis buffer (200 mM KOH and Oligo mix containing 0.2 μM of ATCTGTCTATTTCGTTCATC-7xG4Tag (SEQ ID NO:36), 7xHA-CCAATGCTTA ATCAGTGAG (SEQ ID NO:37), -7xG4Tag, 7xFLAG-CACCAGAATCAGTGCACA AC (SEQ ID NO:38) and 0.01 μM of Positive Control Oligo GTTGGCAAAGGTGAAGAACTGTTTACCGG CGTTGTGCACTGATTCTGGTG) (SEQ ID NO:40), after mix 20 μl of neutralization buffer (30 mM Tris Ph8, 0.5% Tween, complemented with 200 mM HCL). 20 μL of sample mix was loaded on the lateral flow strip (position S FIG. 6), subsequently 40 μL of running buffer (PBS with 0.5% Tween-20) were added in each position B. The present of Ampicillin resistance gene is detected by the accumulation of colloidal Gold at the test line.

1. A labelled protein construct comprising a region that is specific for a target and a plurality of affinity binding sites that are specific to detectable nanoparticles, the construct having at least two separate affinity binding sites independently linked to separate detectable nanoparticles.

2. The labelled protein construct according to claim 1 wherein the plurality of affinity binding sites are selected from the group consisting of His, FLAG, E-tag HA, Strept-tag, myc, S-tag, SH3, and G4T.

3. The labelled construct according to claim 1, wherein the plurality of detectable nanoparticles are antibody coated and said antibodies are linked to the affinity binding sites such that each protein molecule is linked to a plurality of detectable nanoparticles.

4. The labelled construct according to claim 1 comprising a region that is specific for a target and a plurality of affinity binding sites that are specific to particular markers, the markers having further affinity binding sites which may be the same or different, the construct having at least two separate affinity binding sites independently linked to separate detectable nanoparticles in a branched configuration.

5. The labelled construct according to claim 1, wherein there are greater than 50 detectable nanoparticles.

6. The labelled construct according to claim 1, wherein there are between 100 and 10000 detectable nanoparticles.

7. The labelled construct according to claim 6, wherein there are between 500 and 5000 detectable nanoparticles.

8. The labelled construct according to claim 1, wherein the detectable nanoparticles are gold nanoparticles.

9. The labelled construct according to claim 1, wherein the detectable nanoparticles are carbon nanoparticles.

10. The labelled construct according to claim 1, wherein the detectable nanoparticles are silver nucleated gold nanoparticles.

11. The labelled construct according to claim 1, wherein the detectable nanoparticles are platinum nanoparticles.

12. The labelled construct according to claim 1, wherein the detectable nanoparticles are cellulose nanobeads.

13. The labelled construct according to claim 1, wherein the particles are between 5 and 20 nm in size.

14. The labelled construct according to claim 1, wherein the target is selected from the group consisting of a eukaryotic source, a microorganism, a virus, and a microbiome.

15. The labelled construct according to claim 14, wherein the target is a protein.

16. The labelled construct according to claim 14, wherein the target is a nucleic acid.

17. A lateral flow device for detecting the presence of a target in a sample, comprising:
(a) a sample loading area;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area.

18. The device of claim 17, wherein the lateral flow device comprises a solid support selected from the group consisting of glass, paper, nitrocellulose and thread.

19. The device of claim 17, wherein the target is selected from the group consisting of a eukaryotic source, a microorganism, a virus, and a microbiome.

20. The device of claim 19, wherein the eukaryotic source is selected from the group consisting of algae, protozoa, fungi, slime molds and mammalian cells.

21. A method for detecting the presence of a target in a biological sample from a subject, comprising:
i) adding the sample to a sample loading area of a lateral flow device, wherein said device comprises:
(a) a sample loading area;
(b) an area comprising a labelled protein construct comprising a region that is specific for a target in the sample and a plurality of regions that are specific to detectable nanoparticles, the construct having at least two separate affinity binding sites independently linked to separate detectable nanoparticles, wherein the construct is capable of wicking across at least a portion of the lateral flow device;
(c) an area comprising capture probes for the specific target, wherein said capture probes are immobilized on the lateral flow device; and
(d) absorbent material, wherein the absorbent material wicks an aqueous sample across the lateral flow device when the aqueous sample is added to the sample loading area; and
ii) determining from the presence of detectably labelled probe in (c) the presence of the target in the sample.

22. The method according to claim 21, wherein the biological sample from the subject is selected from the group consisting of stool, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool water, pancreatic juice, lavage fluids from sinus cavities, bronchopulmonary aspirates, blastocyl cavity fluid, and umbilical cord blood.

23. A device for detecting the presence of a target analyte in a fluid, the device comprising:
i) a displacement area having a first immobilised marker, for example having a protease or other enzyme, which can be displaced by the presence of the target analyte to produce a first released marker, wherein the displacement occurs without breaking covalent bonds, for example by denaturing a nucleic acid duplex;
ii) one or more signal amplification areas having further immobilised markers which can be released by the presence of the first released (enzymatic) marker to produce a detectable marker, wherein the detectable marker is a labelled protein construct comprising a region that is specific for a target in the sample and a plurality of regions that are specific to detectable nanoparticles, the construct having at least two separate affinity binding sites independently linked to separate detectable nanoparticles; and
iii) one or more detection areas which can identify the presence of the detectable marker;
wherein the displacement area, signal amplification area(s) and detection area(s) are connected such that fluid can flow from the displacement area through the signal amplification area(s) and into the detection area(s).


SEQUENCE LISTING (41 sequences)

SEQ ID NO: 1 - protein, 6 aa, Artificial Sequence
  His tag
  BINDING (1)..(6)
HHHHHH

SEQ ID NO: 2 - protein, 8 aa, Artificial sequence
  FLAG tag
  Binding (1)..(8): Peptide Sequence
DYKDDDDK

SEQ ID NO: 3 - protein, 13 aa, Artificial Sequence
  E-tag
  Binding (1)..(13): Peptide Sequence
GAPVPYPDPLEPR

SEQ ID NO: 4 - protein, 9 aa, Homo sapiens
  Binding (1)..(9): Peptide Sequence
YPYDVPDYA

SEQ ID NO: 5 - protein, 8 aa, Artificial Sequence
  Strept-tag
  Binding (1)..(8): Peptide Sequence
WSHPQFEK

SEQ ID NO: 6 - protein, 10 aa, Homo sapiens
  Binding (1)..(10): Peptide Sequence
EQKLISEEDL

SEQ ID NO: 7 - protein, 15 aa, Bos Taurus
  Binding (1)..(15): Peptide Sequence
KETAAAKFERQHMDS

SEQ ID NO: 8 - protein, 13 aa, Chikungunya virus
  Binding (1)..(13): Peptide Sequence
STVPVAPPRRRRG

SEQ ID NO: 9 - protein, 19 aa, Saccharomyces cerevisiae
  Binding (1)..(19): Peptide Sequence
EELLSKNYHLENEVARLKK

SEQ ID NO: 10 - protein, 13 aa, Artificial Sequence
  Alfa-tag
  Binding (1)..(13): Peptide Sequence
SRLEEELRRRLTE

SEQ ID NO: 11 - protein, 15 aa, Artificial Sequence
  Avi tag
  Binding (1)..(15): Peptide Sequence
GLNDIFEAQKIEWHE

SEQ ID NO: 12 - protein, 4 aa, Artificial Sequence
  C-tag
  Binding (1)..(4): Peptide Sequence
EPEA

SEQ ID NO: 13 - protein, 26 aa, Saccharomyces cerevisiae
  Binding (1)..(26): Peptide Sequence
KRRWKKNFIAVSAANRFKKISSSGAL

SEQ ID NO: 14 - protein, 23 aa, Artificial Sequence
  DogTag
  Binding (1)..(23): Peptide Sequence
DIPATYEFTDGKHYITNEPIPPK

SEQ ID NO: 15 - protein, 16 aa, Artificial Sequence
  IsoPeptag
  Binding (1)..(16): Peptide Sequence
TDKDMTITFTNKKDAE

SEQ ID NO: 16 - protein, 18 aa, Artificial Sequence
  NE-tag
  Binding (1)..(18): Peptide Sequence
TKENPRSNQEESYDDNES

SEQ ID NO: 17 - protein, 7 aa, Artificial Sequence
  Polyglutamate tag
  Binding (1)..(7): Peptide Sequence
EEEEEEE

SEQ ID NO: 18 - protein, 7 aa, Artificial Sequence
  Poly Arginine tag
  Binding (1)..(7): Peptide Sequence
RRRRRRR

SEQ ID NO: 19 - protein, 9 aa, Hippopotamus amphibius
  Binding (1)..(9): Peptide Sequence
TETSQVAPA

SEQ ID NO: 20 - protein, 38 aa, Homo sapiens
  Binding (1)..(38): Peptide Sequence
MDEKTTGWRGGHVVEGLAGELEQLRARLEHHPQGQREP

SEQ ID NO: 21 - protein, 13 aa, Streptococcus dysgalactiae
  Binding (1)..(13): Peptide Sequence
DPIVMIDNDKPIT

SEQ ID NO: 22 - protein, 12 aa, Streptococcus pneumoniae
  Binding (1)..(12): Peptide Sequence
KLGDIEFIKVNK

SEQ ID NO: 23 - protein, 13 aa, Escherichia coli
  Binding (1)..(13): Peptide Sequence
SLAELLNAGLGGS

SEQ ID NO: 24 - protein, 8 aa, Artificial Sequence
  From Rotaria sp. Silwood1 with S to T mutation
  Binding (1)..(8): Peptide Sequence
TQDPSRVG

SEQ ID NO: 25 - protein, 12 aa, Artificial Sequence
  Spot-Tag
  Binding (1)..(12): Peptide Sequence
PDRVRAVSHWSS

SEQ ID NO: 26 - protein, 13 aa, Artificial Sequence
  SpyTag 4MLI_B
  Binding (1)..(13): Peptide Sequence
AHIVMVDAYKPTK

SEQ ID NO: 27 - protein, 11 aa, Plasmodium falciparum
  Binding (1)..(11): Peptide Sequence
MASMTGGQQMG

SEQ ID NO: 28 - protein, 10 aa, Unknown
  SEQ ID 17 from US6610504
  Binding (1)..(10): Peptide Sequence
EVHTNQDPLD

SEQ ID NO: 29 - protein, 6 aa, Artificial Sequence
  Tetra cysteine tag TC-tag
  Binding (1)..(6): Peptide Sequence
CCPGCC

SEQ ID NO: 30 - protein, 11 aa, Vesicular stomatitis virus
  Binding (1)..(11): Peptide Sequence
YTDIEMNRLGK

SEQ ID NO: 31 - protein, 8 aa, Bacteriophage T7
  Binding (1)..(8): Peptide Sequence
DLYDDDDK

SEQ ID NO: 32 - protein, 15 aa, Artificial Sequence
  7x Myc-tag
  Binding (6)..(15): Peptide Sequence
GSKSGEQKLISEEDL

SEQ ID NO: 33 - protein, 17 aa, Artificial Sequence
  Thiolated SH3 peptide from Chikungunya virus
  Binding (1)..(17): Peptide Sequence
MGSGSTVPVAPPRRRRG

SEQ ID NO: 34 - protein, 24 aa, Artificial Sequence
  GCN4p tag derived from Saccharomyces cerevisiae GCN4p
  Binding (1)..(24): Peptide Sequence
GSKSGEELLSKNYHLENEVARLKK

SEQ ID NO: 35 - protein, 18 aa, Artificial Sequence
  Cysteine SH3 from Chikungunya virus
  Binding (1)..(18): Peptide Sequence
MCGSGSTVPVAPPRRRRG

SEQ ID NO: 36 - DNA, 20 bp, Artificial Sequence
  Olignoucleotide Sequence with 3′ thiol group
  Unsure (1)..(20)
atctgtctatttcgttcatc

SEQ ID NO: 37 - DNA, 19 bp, Artificial Sequence
  Oligonucleotide Sequence with 5′ thiol group
  Unsure (1)..(19)
ccaatgcttaatcagtgag

SEQ ID NO: 38 - DNA, 21 bp, Artificial Sequence
  Oligonucleotide Sequence with 3′ thiol group
  Unsure (1)..(21)
cagttcttcacctttgccaac

SEQ ID NO: 39 - DNA, 20 bp, Artificial Sequence
  Oligonucleotide Sequence with 5′ thiol group
  Unsure (1)..(20)
caccagaatcagtgcacaac

SEQ ID NO: 40 - DNA, 50 bp, Artificial Sequence
  Oligonucleotide Sequence
  Unsure (1)..(50)
gttggcaaaggtgaagaactgtttaccggcgttgtgcactgattctggtg

SEQ ID NO: 41 - DNA, 62 bp, Unknown
  Oligonucleotide sequence
tggatgaacgaaatagacagatcgctgagataggtgcctcactgattaagcattggtaaggt